(19) United States
(12) Patent Application Publication
(10) Pub. No.: US 2011/0054384 A1
(43) Pub. Date: Mar. 3, 2011

(54) Sonic Device for Use in Capsule of Eye

(76) Inventor: David C. Brown, Fort Myers, FL (US)

(21) Appl. No.: 12/547,609

(22) Filed: Aug. 26, 2009

(51) Int. Cl. A61F 9/007

(52) U.S. Cl. 604/22

(57) ABSTRACT

This invention relates to an improved procedure for polishing the interior of the lens capsule after the lens is removed. The procedure includes removing the phacoemulsification instrument from the eye after the lens and cortex material have been removed and then placing a novel sonic tip over or within the needle portion of the instrument. The tip portion of the instrument is then reinserted into the lens capsule with a lower ultrasonic energy level. The sonic tip is then placed in close proximity or in direct contact with the interior surface of the lens capsule to remove any remaining small particles of the cortex or other cells.

### BACKGROUND OF INVENTION

1. Field of the Invention

This invention relates to eye surgery procedures and particularly to a technique for removing unwanted material from the lens capsule of the eye after the natural lens has been removed. Cortex material and epithelial cells remaining after the lens has been removed need to be removed to the maximum extent possible. This invention relates to an improved technique for removing these materials from the lens capsule and to a specially designed sonic tip for attachment to a conventional phacoemulsification instrument.

2. Description of Related Art

Currently during cataract surgery, a conventional phacoemulsification instrument as illustrated in FIG. 1 is used to apply ultrasonic energy to the lens, which breaks up or emulsifies the cataract. An aspiration passageway 15 carries away the fragmented portions of the cataract and irrigation passageway 12 provides an irrigating fluid to the interior of the lens capsule to clear out the fragments and to prevent the lens capsule from collapsing. In addition to removing the lens it is desirable to remove cortex material and epithelial cells from the lens capsule prior to insertion of the replacement lens. This procedure is commonly called “irrigation and aspiration.” The lens capsule also harbors germinal and lens epithelial cells, which may be removed by “polishing” the capsule.

While conventional needles of the phacoemulsification instruments are typically formed with a sharp edge as shown in FIG. 1, some prior art needles have curved or ball shaped end portions as shown in U.S. Pat. No. 7,204,820. It is also known that by lowering the ultrasonic energy level transmitted to the surface of the tip portion of a phacoemulsification hand piece, the surface can be used to selectively clean tissue without the capsule being broken up. Such is disclosed in the U.S. Patent to Struchel et al, U.S. Pat. No. 6,159,175.

### BRIEF SUMMARY OF THE INVENTION

The present invention provides an apparatus and a method of improving the amount of unwanted matter, cortex and epithelial and other intracapsular cells, removed from the lens capsule after the natural lens has been removed, without undue delay. This is accomplished by providing a novel sonic cleaning tip that can be attached to the distal portion of a conventional phacoemulsification instrument after the instrument has been used to remove the natural cataract. The amount of energy applied to the tip by the instrument is reduced so as not to cause emulsification of eye tissue, and the tip of the instrument is then relocated into the lens capsule.

### BRIEF DESCRIPTION OF THE SEVERAL VIEWS OF THE DRAWING(S)

FIG. 1 is a sectional view of a conventional phacoemulsification instrument.

FIG. 2 is a sectional view of one embodiment of the present invention.

FIG. 3 is a sectional view of a second embodiment of the invention.

FIG. 4 is a sectional view of a third embodiment of the invention.

FIG. 5 is a sectional view of a fourth embodiment of the invention.

FIG. 6 is a sectional view of yet another embodiment of the invention.

FIG. 7 is a sectional view of a further embodiment of the invention.

FIG. 8 is a sectional view of a variation of the embodiment of FIG. 5.

FIG. 9 is a sectional view of a variation of the embodiment of FIG. 6.

### DETAILED DESCRIPTION OF THE INVENTION

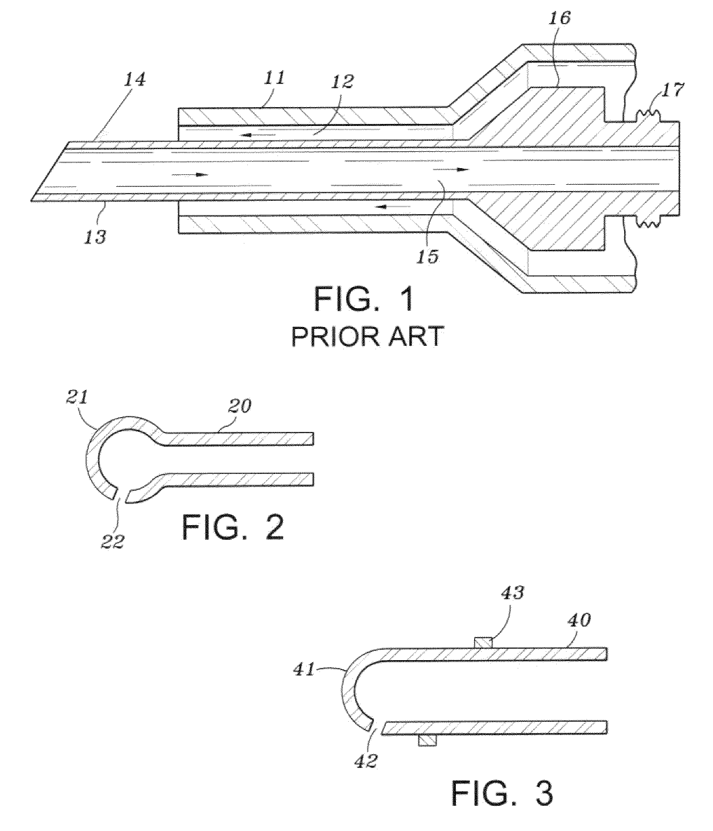

Referring to FIG. 1, a conventional phacoemulsification instrument is shown, including an outer sleeve 11, an inner hollow needle 14 and a threaded shoulder 17 for attachment to a source of ultrasonic energy. Passageway 15 serves as an aspiration conduit and the annular space 12 between outer sleeve 11 and needle 14 serves as an irrigation conduit for a suitable liquid. This instrument is sufficient for removing the natural lens and a major portion of the cortex material, however tiny particles of the cortex and epithelial cells are left behind. The present invention envisions the use of a specially designed sonic tip to be placed either over or within the terminal portion of a conventional needle 14 of the instrument.

Once the natural lens and the majority of the cortex material is removed, the surgeon withdraws the tip portion of the hand piece from the eye and in accordance with the invention places one of the sonic tips over or within the hollow needle. The distal portion of the hand piece with the sonic tip attached thereto is reinserted into the lens capsule. The power level and vacuum of the ultrasonic generator is reduced such that emulsification or capsule tear does not occur. The curved or rounded portion of the sonic tip can engage or be placed in close proximity to the inner surface of the lens capsule to remove any remaining cortex particles and cells.

FIG. 2 illustrates one embodiment of a sonic tip. The tip includes a tubular portion 20 that has an outer diameter that would tightly engage the inner surface of the hollow needle 13. Alternatively the diameter of the inner section of the tubular portion 20 could be selected to slide over the outer surface of the hollow needle. The distal end of the sonic tip is formed as a semi-spherical hollow body 21. An orifice 22 is provided through the wall of the hollow body to provide a passageway for aspirating fluid.

A second embodiment of the sonic tip is shown in FIG. 3. In this case, a hollow circular tubular member 40 terminates in a rounded surface at 41. An orifice 42 is provided for the aspirating fluid. The inner and outer diameters of the hollow tube can be selected to either fit over the end of needle 14 or fit within the hollow portion of the needle. In the latter case, an annular shoulder 43 complimentary to the shape of the end of the needle may be provided as a stop. Alternatively, the shoulder could be formed on the interior surface when the tip is positioned over needle portion 13.

In FIG. 4 a third embodiment is shown. The shape of the sonic tip in this case has a bulbous section 31 extending outwardly from the longitudinal axis of tubular member 33. An orifice 32 is provided in the bulbous section 31. A stop shoulder 34 may also be provided on the exterior surface in the situation where the outer diameter of tubular member 33 is chosen so that the tip can be slidally received within the hollow needle.

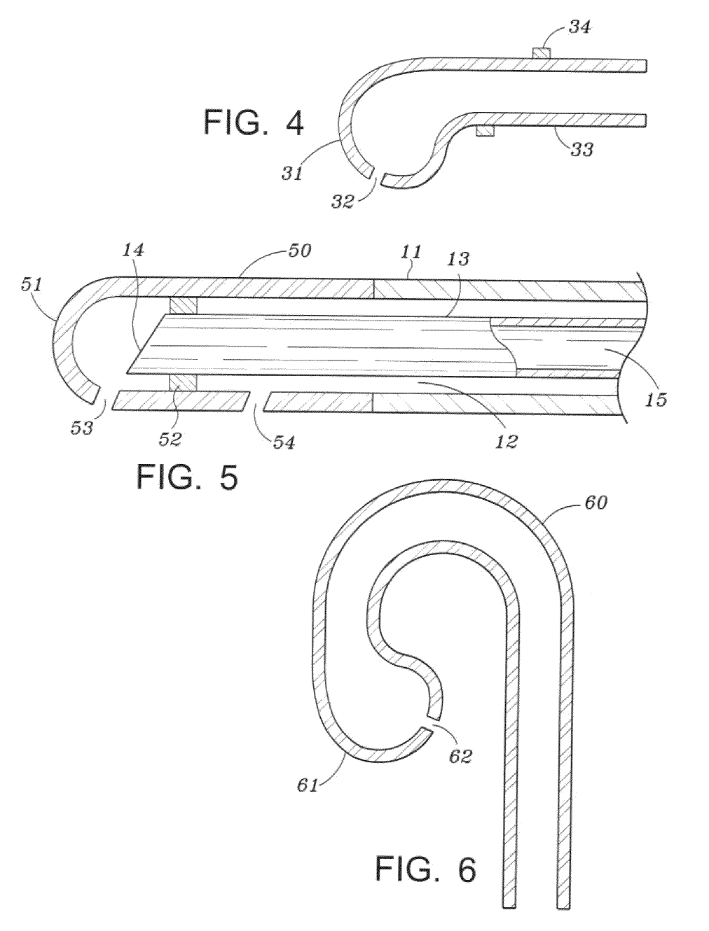

FIG. 5 represents a fourth embodiment of the invention. The sonic tip is provided with a seal 52 that spaces the inner surface of the tubular member 50 from the outer surface 13 of the needle. Tubular member 50 has a distal semi-circular end portion 51 with an orifice 53 to accommodate the aspiration fluid. A suitable aperture 54 is provided through tubular member 50 upstream of the seal 52. In this embodiment irrigation fluid flows through passageway 12 and out aperture 54 while orifice 53 and passageway 15 form a return path for the aspiration fluid. Alternatively the entire outer sleeve 11 could be removed and replaced with a sleeve that has sonic tip 50 formed as an integral portion of the sleeve.

In the embodiment of FIG. 6, the sonic tip 60 is formed as a U-shaped tip. As in previous embodiments, the diameter of the tubular portion may be chosen to fit within or slide over needle 14. Also an inclined annular stop can be provided on the interior or exterior surface of the tube as discussed earlier. The sonic tip of the present invention can be made of any solid material that serves as a transmission medium for the ultrasonic energy. Suitable materials include nylon, silicone, acrylic, PMMA, metal, or other rigid material, for example.

In the embodiment of FIG. 7, the tip has the shape of a spatula having a flat surface 71 leading from tubular section 70. The orifice 22 is located in the end portion of tubular member 70. Tubular portion 70 may be sized to fit within or over needle 13.

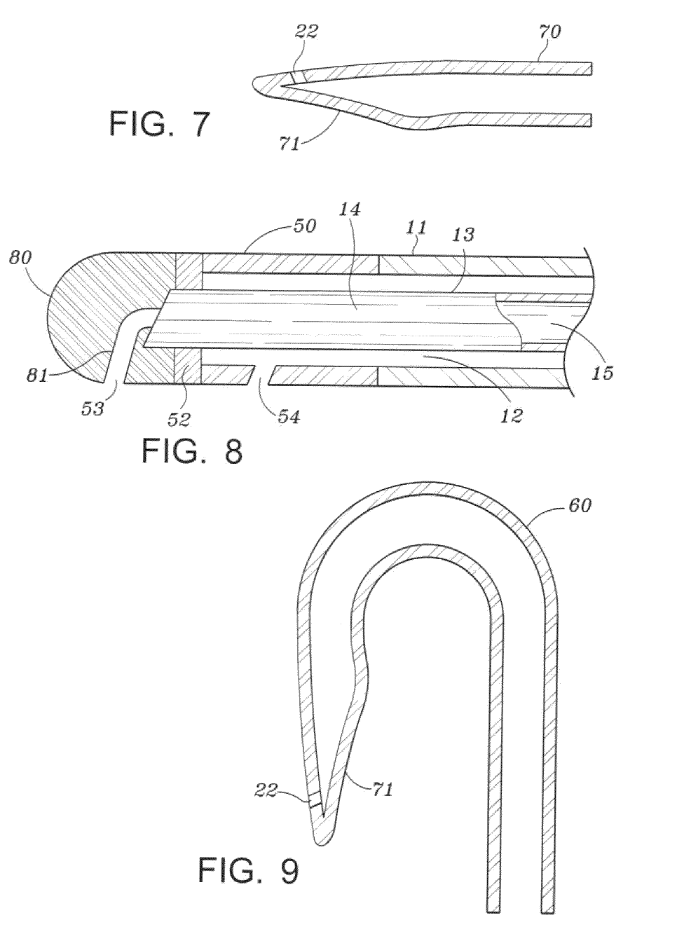

FIG. 8 illustrates a variation of the embodiment of FIG. 5. In the embodiment, the sonic tip consists of a solid tip 80 of soft material. An aspiration passageway 81 extends between the exterior of the tip and aspiration conduct 15 of the instrument. The ultrasonic needle 14 abuts the solid tip and transfers energy to the tip.

The embodiment of FIG. 9 is similar to that of FIG. 6 except for the shape of the tip portion. The tubular portion 90 is U-shaped while the end portion 91 has the shape of a spatula similar to FIG. 7. The tip is provided with an aspiration orifice 22 as in the other embodiments.

Although the present invention has been described with respect to specific details, it is not intended that such details should be regarded as limitations on the scope of the invention, except to the extent that they are included in the accompanying claims.

I claim:

1. A sonic tip for placement on or within the needle portion of a phacoemulsification hand piece comprising:
a tubular member having a first end portion and a second portion end, the first end portion of the tubular member being open, the second portion end of the tubular member have a closed end, an outside surface of the closed end having a smooth curved surface, and an orifice extending through the closed end curved surface.

2. A sonic tip according to claim 1 wherein the curved surface is in the shape of a sphere.

3. A sonic tip according to claim 1 wherein the curved surface is bulbous in shape.

4. A sonic tip according to claim 1 wherein the tubular member is u-shaped.

5. A sonic tip according to claim 1 including an inclined, annular stop member on the inner or outer surface of the tubular member.

6. A sonic tip according to claim 1 including an annular sealing ring on interior surface of the tubular member and a port in the wall of the tubular member near the open end.

7. A sonic tip according to claim 1 wherein the tip is made of nylon.

8. A sonic tip according to claim 1 wherein the closed end is in the shape of a spatula.

9. A sonic tip according to claim 4 wherein the closed end is in the shape of a spatula.

10. A sonic tip according to claim 6 wherein the second end portion comprises a solid, soft material with a passageway extending from the outside surface to the interior of the tubular member.

11. A method of polishing the interior surface of a lens capsule following removal of the lens using a phacoemulsification instrument that has a needle portion comprising:
a) removing the phacoemulsification instrument from the eye after the lens has been removed,
b) placing a sonic tip either over or within a needle portion of the instrument,
c) setting the energy level of the ultrasonic power source below the value required to emulsify the lens of the human eye,
d) inserting the tip portion of the instrument with the sonic tip into the lens capsule, and
e) positioning the sonic tip in close proximity to or in contract with the interior surface of the lens capsule.

12. The method of claim 8 including providing a flow of irrigating fluid to the lens capsule and aspirating the fluid from the lens capsule.
